(19) United States
(12) Patent Application Publication
(10) Pub. No.: US 2026/0357315 A1
(43) Pub. Date: Nov. 12, 2026

(54) PARALLELIZED ELECTROCHEMICAL POLYNUCLEOTIDE SYNTHESIS

(71) Applicant: Kern Systems, Inc., Allston, MA (US)

(72) Inventors: Pradeep BUGGA, Charlestown, MA (US); Henry Hung-Yi LEE, Brookline, MA (US)

(21) Appl. No.: 19/636,153

(22) Filed: Apr. 1, 2026

Related U.S. Application Data
(63) Continuation of application No. PCT/US2024/050375, filed on Oct. 8, 2024.
(60) Provisional application No. 63/589,114, filed on Oct. 10, 2023.

(51) Int. Cl. C12P 19/34; C12N 9/12; C25B 3/09; C25B 11/042; C25B 15/025; C25B 15/08

(52) U.S. Cl. CPC C12P 19/34; C12N 9/1241; C25B 3/09; C25B 11/042; C25B 15/025; C25B 15/08

(57) ABSTRACT

The present disclosure provides methods and devices for synthesis of nucleic acid molecules. Devices of the present disclosure may include a substrate, a first electrode, a second electrode, and a plurality of initiator molecules. Methods of the present disclosure may include providing a template-independent DNA polymerase to the device. The template-independent DNA polymerase may be usable to extend the initiator molecules, thereby synthesizing nucleic acid molecules. The first and second electrode may be usable modulate or otherwise control the activity of the template-independent DNA polymerase.

### CROSS-REFERENCE

This application is a continuation application of International Application No. PCT/US2024/050375, filed Oct. 8, 2024, which claims the benefit of U.S. Provisional Application No. 63/589,114, filed Oct. 10, 2023, each of which is entirely incorporated herein by reference.

### BACKGROUND

The use of synthesized polynucleotides is increasing across a variety of fields, including, for example, medicine, diagnostics, and data storage. Polynucleotides may be synthesized using chemical (e.g., phosphoramidite) or enzymatic (e.g., polymerase extension) processes. Parallelization of synthesis methods may reduce cost and increase efficiency of polynucleotide synthesis.

### SUMMARY

The present disclosure provides devices and methods for parallelized enzymatic polynucleotide synthesis using electrode arrays. The devices and methods described herein may provide for electrical control of enzyme activity to permit large-scale synthesis of unique oligonucleotide or polynucleotide molecules. Large-scale synthesis of unique oligonucleotide or polynucleotide molecules may increase synthesis efficiency and reduce costs associated with synthesis. Increasing efficiency and reducing cost of oligonucleotide and polynucleotide synthesis may permit the use of polynucleotides in fields in which synthesis is prohibitively expensive, such as, for example, nucleic acid-based data storage.

In an aspect, the present disclosure provides a device for generating polynucleotide sequences, comprising: a substrate configured to contact a solution comprising a template-independent deoxyribonucleic acid (DNA) polymerase; a first electrode disposed on a surface of the substrate, wherein the first electrode is configured to be in electrical communication with the solution; a second electrode disposed on the surface of the substrate, wherein the second electrode is configured to be in electrical communication with the solution, wherein the first electrode and the second electrode are configured to generate a local pH change to the solution in a reaction site adjacent to the first electrode upon application of an electrical potential to the first electrode or the second electrode, and wherein, during use, the local pH change activates the template-independent DNA polymerase disposed in the reaction site; and a plurality of initiator oligonucleotides immobilized on the first electrode by a plurality of linker molecules such that, during use, the plurality of initiator oligonucleotides is disposed in the reaction site, and wherein the plurality of initiator oligonucleotides is usable by the template-independent DNA polymerase to generate the polynucleotide sequences.

In some embodiments, a linker molecule of the plurality of linker molecules comprises a spacer. In some embodiments, a length of the spacer is greater than or equal to about 1.5 nanometers (nm). In some embodiments, the length of the spacer is less than or equal to about 8 nm. In some embodiments, a linker molecule of the plurality of linker molecules comprises a cleavage site. In some embodiments, the cleavage site comprises an uracil nucleobase. In some embodiments, the cleavage site is configured to be cleaved upon application of an ultra-violet (UV) light, enzyme, electricity, heat, pH, or any combination thereof. In some embodiments, the plurality of linker molecules comprise one or more members selected from the group consisting of polyethylene glycol (PEG) 200, PEG500, PEG1000, PEG2000, 11-mercaptoundecanoic acid, heterobifunctional PEG linkers, and any combination thereof.

In some embodiments, the first electrode comprises one or more members selected from the group consisting of gold, aluminum, ruthenium, tungsten, silicon, and any combination thereof. In some embodiments, the plurality of linker molecules comprises thiol-functionalization groups. In some embodiments, the plurality of linker molecules comprises aryldiazonium functionalization group. In some embodiments, the plurality of linker molecules is coupled to the first electrode via peptide coupling, alkyne-azide coupling, thiol-maleimide coupling, biotin-streptavidin coupling, or any combination thereof. In some embodiments, the device further comprises a third electrode configured to measure a response voltage or current of the solution in the reaction site.

In some embodiments, the device comprises a plurality of first electrodes comprising the first electrode and a plurality of second electrodes comprising the second electrode. In some embodiments, the first electrode and second electrode of the reaction site are individually addressable from another first electrode and another second electrode of another reaction site. In some embodiments, the device further comprises circuitry configured to couple to an electroanalytical device.

In some embodiments, the device further comprises a flow cell configured to receive the solution and direct the solution to the substrate. In some embodiments, an initiator oligonucleotide of the plurality of initiator oligonucleotides comprises at least three nucleobases. In some embodiments, an initiator oligonucleotide of the plurality of initiator oligonucleotides is coupled to the first electrode via a 5′ end. In some embodiments, the first electrode and the second electrodes are planar electrodes. In some embodiments, the second electrode is associated with more than one first electrode.

In another aspect, the present disclosure proves a device for generating polynucleotide sequences, comprising: a substrate configured to contact a solution comprising a template-independent DNA polymerase; a first electrode disposed on a surface of the substrate, wherein the first electrode is configured to be in electrical communication with the solution, wherein the first electrode is configured to generate a local pH change to the solution in a reaction site adjacent to the first electrode upon application of an electrical potential to the first electrode, and wherein, during use, the local pH change activates the template-independent DNA polymerase disposed in the reaction site; a second electrode disposed on the surface of the substrate and at least partially circumscribing the first electrode, wherein the second electrode is configured to be in electrical communication with the first electrode and the solution, and wherein the second electrode is configured to confine the local pH change to the reaction site; and a plurality of initiator oligonucleotides immobilized in the reaction site, wherein the plurality of initiator oligonucleotides is usable by the template-independent DNA polymerase to generate the polynucleotide sequences.

In some embodiments, the device further comprises a third electrode configured to measure a response voltage or current of the solution in the reaction site. In some embodiments, the third electrode at least partially circumscribes the first electrode. In some embodiments, the device comprises a plurality of first electrodes comprising the first electrode and a plurality of second electrodes comprising the second electrode. In some embodiments, the first electrode and second electrode of the reaction site are individually addressable from another first electrode and another second electrode of another reaction site.

In some embodiments, the device further comprises circuitry configured to couple to an electroanalytical device. In some embodiments, the device further comprises a flow cell configured to receive the solution and direct the solution to the substrate. In some embodiments, an initiator oligonucleotide of the plurality of initiator oligonucleotides comprises at least three nucleobases. In some embodiments, an initiator oligonucleotide of the plurality of initiator oligonucleotides is coupled to the reaction site via a 5′ end. In some embodiments, the first electrode and the second electrodes are planar electrodes.

In another aspect, the present disclosure provides a method for generating polynucleotide sequences, comprising: (a) providing a device comprising (i) a substrate in contact with a solution comprising a template-independent DNA polymerase, (ii) a first electrode disposed on a surface of the substrate and in electrical communication with the solution, (iii) a second electrode disposed on the surface of the substrate and in electrical communication with the solution and the first electrode, and (iv) a plurality of initiator oligonucleotide immobilized on the first electrode by a plurality of linker molecules such that the plurality of initiator oligonucleotides is disposed in a reaction site adjacent to the first electrode; (b) applying an electrical potential to the first electrode or the second electrode to generate a local pH change to the solution in the reaction site, wherein the local pH change activates the template-independent DNA polymerase disposed in the reaction site; and (c) using the plurality of initiator oligonucleotides and the template-independent DNA polymerase disposed in the reaction site to generate the polynucleotide sequences.

In some embodiments, the plurality of linker molecules comprises a plurality of spacers. In some embodiments, a length of a spacer of the plurality of spacers is greater than or equal to about 1.5 nm. In some embodiments, the length of the spacer is less than or equal to about 8 nm. In some embodiments, the method further comprises, prior to (a), using electrografting to immobilize the plurality of initiator oligonucleotides to the first electrode. In some embodiments, an initiator oligonucleotide of the plurality of initiator oligonucleotides comprises at least three nucleobases.

In some embodiments, the method further comprises providing a plurality of nucleobases to the substrate, wherein in (c), the template-independent DNA polymerase couples nucleobases of the plurality of nucleobases to the plurality of initiator oligonucleotides disposed in the reaction site. In some embodiments, prior to (b), the template-independent DNA polymerase is inactive. In some embodiments, the method further comprises, subsequent to (b), halting application of the voltage or current the first electrode and the second electrode, wherein the halting inactivates the template-independent DNA polymerase. In some embodiments, the method further comprises, prior to (a), generating the first and the second electrode via screen printing. In some embodiments, the solution comprises a redox agent and, wherein, during (b), application of the voltage of electrode generates an oxidation reaction with the redox agent that generates the local pH change. In some embodiments, the redox agent comprises tetramethylquinone, tetramethylhydroquinone, or a combination thereof.

In another aspect, the present disclosure provides a method for generating polynucleotide sequences, comprising: (a) providing a device comprising (i) a substrate in contact with a solution comprising a template-independent DNA polymerase, (ii) a first electrode disposed on a surface of the substrate and in electrical communication with the solution, (iii) a second electrode disposed on the surface of the substrate and in electrical communication with the solution and the first electrode, and (iv) a plurality of initiator oligonucleotide immobilized to a reaction site adjacent to the first electrode, wherein the second electrode at least partially circumscribes the first electrode; (b) applying a first electrical potential to the first electrode to generates a local pH change to the solution in the reaction site, wherein the local pH change activates the template-independent DNA polymerase disposed in the reaction site; (c) applying a second electrical potential to the second electrode to confine the local pH change to the reaction site; and (d) using the plurality of initiator oligonucleotides and the template-independent DNA polymerase disposed in the reaction site to generate the polynucleotide sequences.

In some embodiments, an initiator oligonucleotide of the plurality of initiator oligonucleotides comprises at least three nucleobases. In some embodiments, the method further comprises providing a plurality of nucleobases to the substrate, wherein in (c), the template-independent DNA polymerase couples nucleobases of the plurality of nucleobases to the plurality of initiator oligonucleotides disposed in the reaction site. In some embodiments, prior to (b), the template-independent DNA polymerase is inactive. In some embodiments, the method further comprises, subsequent to (b), halting application of the voltage or current the first electrode and the second electrode, wherein the halting inactivates the template-independent DNA polymerase. In some embodiments, the method further comprises, prior to (a), generating the first and the second electrode via screen printing. In some embodiments, the solution comprises a redox agent and, wherein, during (b), application of the voltage of electrode generates an oxidation reaction with the redox agent that generates the local pH change. In some embodiments, the redox agent comprises tetramethylquinone, tetramethylhydroquinone, or a combination thereof. In some embodiments, the method further comprises using a third electrode to measure a response voltage or current of the solution in the reaction site. In some embodiments, the third electrode at least partially circumscribes the first electrode.

In another aspect, the present disclosure provides a device for generating polynucleotide sequences, comprising: a substrate configured to contact a solution comprising a template-independent deoxyribonucleic acid (DNA) polymerase; a first electrode disposed on a surface of the substrate, wherein the first electrode is configured (i) to immobilize initiator oligonucleotides usable by the template-independent DNA polymerase to generate the polynucleotide sequences and (ii) to generate a local pH change to the solution disposed at a reaction site adjacent to the first electrode upon application of a first electrical potential to the first electrode; a second electrode disposed on the surface of the substrate adjacent to the first electrode, wherein, during use, the second electrode is configured to confine the local pH change to the reaction site upon application of a second electrical potential to the second electrode; a third electrode disposed on the surface of the substrate adjacent to the first electrode and the second electrode, wherein, during use, the third electrode is configured to permit measurement of current or voltage adjacent to the first electrode; and a flow cell configured to deliver the solution comprising the template-independent DNA polymerase to the substrate.

In some embodiments, the second electrode and the third electrode at least partially circumscribe the first electrode. In some embodiments, the first electrode comprises one or more members selected from the group consisting of gold, aluminum, ruthenium, tungsten, silicon, and any combination thereof. In some embodiments, the device comprises a plurality of first electrodes comprising the first electrode, a plurality of second electrodes comprising the second electrode, and a plurality of third electrode comprising the third electrode. In some embodiments, the first electrode and second electrode of the reaction site are individually addressable from another first electrode and another second electrode of another reaction site. In some embodiments, the device further comprises circuitry configured to couple to an electroanalytical device. In some embodiments, the first electrode, the second electrode, and the third electrode are planar electrodes.

In another aspect, the present disclosure provides a device for large-scale synthesis of a plurality of unique polynucleotide sequences, comprising: a substrate comprising an array of reaction sites, wherein a reaction site of the array of reaction sites comprises a first electrode and a second electrode configured to (i) generate a local pH change and (ii) confine the local pH change to the reaction site upon application of an electrical potential to the first electrode or the second electrode, wherein the local pH change is configured to activate a template-independent deoxyribonucleic (DNA) polymerase, and wherein the reaction site is individually addressable such that the template-independent DNA polymerase is usable to generate the plurality of unique polynucleotide sequences across the array of reaction sites; a plurality of initiator oligonucleotides immobilized to the array of reaction sites, wherein the plurality of initiator oligonucleotides is usable by a template-independent DNA polymerase to generate the plurality of unique polynucleotide sequences; and a flow cell configured to provide the template-independent DNA polymerase to the substrate.

In some embodiments, the plurality of unique polynucleotide sequences is configured for deoxyribonucleic acid data storage. In some embodiments, the substrate has a surface area from about 10 squared millimeters (mm²) to 100 mm². In some embodiments, the device is configured to store at least about 1 million bytes of data. In some embodiments, the device is configured to store at least about 1 billion bytes of data.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic of an example device for nucleic acid synthesis.

FIG. 2 shows example template-independent DNA polymerase activity as a function of linker length.

FIG. 3 shows example template-independent DNA polymerase activity as a function of pH.

FIG. 4 shows example potential response to a reductive current applied to redox agent in a reaction site.

FIG. 5 shows nucleotide extension with and without application of current to the reaction site.

FIG. 6 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “nucleotide,” as used herein, generally refers to an organic molecule that serves as the monomer, or subunit, of a nucleic acid molecule, such as a deoxyribonucleic (DNA) molecule or ribonucleic acid (RNA) molecule. In some embodiments, a nucleic acid molecule may also be a peptide nucleic acid (PNA) nucleotide, a locked nucleic acid (LNA) nucleotide, or a dideoxynucleotide. Such subunit can be A, C, G, T, or U, or any other subunit that is specific to one of more complementary A, C, G, T, or U, or complementary to a purine (e.g., A or G, or variant thereof) or pyrimidine (e.g., C, T, or U, or variant thereof). Nucleotides may be naturally occurring, modified, or a nucleotide analog. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, 2,6-diaminopurine or the like.

The term “oligonucleotide,” as used herein, generally refers to a nucleic acid molecule comprising at least one nucleotide that may have various lengths. Nucleotides that make up an oligonucleotide may be, for example, deoxyribonucleotides or ribonucleotides or analogs thereof. An oligonucleotide may comprise at least about 2, 3, 4, 5, 6, 7, 8, 9, 10, 11, 12, 13, 14, 15, 16, 17, 18, 19, 20, or more nucleotides. An oligonucleotide may comprise at most about 20, 19, 18, 17, 16, 15, 14, 13, 12, 11, 10, 9, 8, 7, 6, 5, 4, 3, 2, or less nucleotides. An oligonucleotide may be unbound (e.g., in solution) or bound (e.g., chemically bonded to a substrate). Oligonucleotides may include one or more nonstandard nucleotide(s), nucleotide analog(s), modified nucleotides, or any combination thereof.

The term “initiator oligonucleotide,” as used herein, generally refers to an oligonucleotide usable as a substrate for a polymerase enzyme to generate a polynucleotide. The initiator oligonucleotide may be usable as a template or substrate for a polymerase enzyme. Alternatively, the initiator oligonucleotide may be a substrate for a template-independent polymerase, such as terminal deoxynucleotidyl transferase (TdT).

The term “polynucleotide,” as used herein, generally refers to a nucleic acid molecule comprising at least 13 nucleotides. A polynucleotide may comprise at least about 13, 14, 15, 16, 18, 20, 25, 30, 35, 40, 45, 50, 60, 70, 80, 90, 100, 125, 150, 175, 200, 250, 300, 400, 500, 600, 700, 800, 900, 1,000, 5,000, 10,000, 50,000, 100,000 or more nucleotides. In some examples, a polynucleotide comprises at most about 100,000, 50,000, 10,000, 5,000, 1,000, 900, 800, 700, 600, 500, 400, 300, 250, 200, 175, 150, 125, 100, 90, 80, 70, 60, 50, 45, 40, 35, 30, 25, 20, 19, 18, 17, 16, 15, 14, or 13 nucleotides. A polynucleotide may be unbound (e.g., in solution) or bound (e.g., chemically bonded to a substrate).

The term “linker molecule,” as used herein, generally refers to any molecule configured to couple or that couples a molecule to a substrate. A linker molecule may have a first reactive moiety configured to couple to or that couples to a substrate. A linker molecule may have a second reactive moiety configured to couple to or that couples to a molecule. The first and the second reactive moieties may be the same type of reactive moieties or may be different types of reactive moieties. A linker molecule may have at least 2, 3, 4, 5, 6, 8, 10, 12, 15, 20, or more reactive moieties. A linker molecule may be any length. A linker molecule may be configured to couple or may couple the molecule to a substrate with minimal spacing between the molecule and the substrate. Alternatively, a linker molecule may be configured to or may space the molecule away from a surface of the substrate. A linker molecule may have any length, for example, a length of greater than or equal to about 0.2 nanometers (nm), 0.4 nm, 0.6 nm, 0.8 nm, 1 nm, 1.2 nm, 1.5 nm, 2 nm, 3 nm, 4 nm, 5 nm, 6 nm, 8 nm, 10 nm, or more. A linker molecule may have any length, for example, a length of less than or equal to about 10 nm, 8 nm, 6 nm, 5 nm, 4 nm, 3 nm, 2 nm, 1.5 nm, 1.2 nm, 1 nm, 0.8 nm, 0.6 nm, 0.4 nm, 0.2 nm, or less.

The term “electrode,” as used herein, generally refers to an electric conductor that carries electric current into non-metallic solids, liquids, gases, plasmas, or vacuums. An electrical conductor may be but is not always a solid. The electrode from which electrons emerge is the cathode and is designated negative while the electrode that receives electrons is the positively designated anode.

As used herein, the term “array” refers to a population of sites that can be differentiated from each other according to relative location. Different molecules that are at different sites (e.g., reaction sites) of an array can be differentiated from each other according to the locations of the sites in the array. An individual site of an array can include one or more molecules (e.g., oligonucleotides or polynucleotides) of a particular type.

The term “distinct,” or “unique,” as used herein, generally refers to an item or object that is different from another item or object. For example, a distinct, or unique, polynucleotide may be a polynucleotide with a sequence that is different from a sequence of any other polynucleotide. A distinct, or unique, oligonucleotide may not have the same sequence as any other oligonucleotide. The distinct, or unique, oligonucleotide or polynucleotide may share regions of similarity with another oligonucleotide or polynucleotide.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The present disclosure provides devices and methods for electrochemical enzymatic synthesis of oligonucleotides or polynucleotides on an electrode or on an electrode array. The devices and methods described herein may provide for parallelized, large-scale polynucleotide synthesis at lower cost and higher efficiencies than other polynucleotide synthesis methods and systems. The electrochemical enzymatic polynucleotide synthesis described herein may provide for de novo synthesis of polynucleotides. Nucleotide incorporation may be modulated or otherwise controlled by the application of an electrical potential to electrodes of a reaction site on an electrode array. Application of an electrical potential to a reaction site on an electrode array may alter the pH at the reaction site. Altering the pH at the reaction site may activate or deactivate a polymerase enzyme. Activation or deactivation of polymerase enzymes may permit selective incorporation of nucleotides into a surface immobilized initiator oligonucleotide to generate a polynucleotide sequence.

The devices and methods described herein may be usable for generating an array of polynucleotide sequences. An array site (e.g., reaction site) may include a distinct or unique polynucleotide sequence such that the sequence of the polynucleotide is different from the sequence of a polynucleotide generated at another array site (e.g., reaction site). The devices and methods described herein may permit control over the sequence of the generated polynucleotides such that a given polynucleotide sequence may be generated at a given reaction site. Such large-scale construction of designed polynucleotides may be useful for nucleic acid-based data storage (e.g., DNA data storage), nucleic acid library generation, protein engineering, DNA nanotechnology, fundamental and applied biological research, and other life science applications.

### Devices for Polynucleotide Synthesis

In an aspect, the present disclosure provides devices for generating oligonucleotide or polynucleotide sequences. A device may include a substrate, first electrode, second electrode, a plurality of initiator oligonucleotides, or any combination thereof. The substrate may be configured to contact or may contact a solution comprising a template-independent deoxyribonucleic acid polymerase. The first electrode may be disposed on a surface of the substrate. The first electrode may be configured to be in electrical communication with or may be in electrical communication with the solution. The second electrode may be disposed on the surface of the substrate. The second electrode may be configured to be in electrical communication with or may be in electrical communication with the solution. The first electrode and the second electrode may be configured to generate or may generate a local pH change to the solution in a reaction site adjacent to the first electrode upon application of an electrical potential to the first electrode or the second electrode. During use the local pH change may activate the template-independent DNA polymerase disposed in the reaction site. The plurality of initiator oligonucleotides may be immobilized on the first electrode by a plurality of linker molecules. During use the plurality of initiator oligonucleotides may be disposed in the reaction site. The plurality of initiator oligonucleotides may be usable by the template-independent DNA polymerase to generate the oligonucleotide or polynucleotide sequence(s).

In another aspect, the present disclosure provides devices for generating oligonucleotide or polynucleotide sequences. A device may include a substrate, first electrode, second electrode, a plurality of initiator oligonucleotides, or any combination thereof. The substrate may be configured to contact or may contact a solution comprising a template-independent deoxyribonucleic acid polymerase. The first electrode may be disposed on a surface of the substrate. The first electrode may be configured to be in electrical communication with or may be in electrical communication with the solution. The first electrode may be configured to generate or may generate a local pH change to the solution in a reaction site adjacent to the first electrode upon application of an electrical potential to the first electrode. During use the local pH change may activate the template-independent DNA polymerase disposed in the reaction site. The second electrode may be disposed on the surface of the substrate. The second electrode may at least partially circumscribe the first electrode. The second electrode may be configured to be in electrical communication with or may be in electrical communication with the first electrode and the solution. The second electrode may be configured to confine or may confine the local pH change to the reaction site. The plurality of initiator oligonucleotides may be immobilized in the reaction site. The plurality of initiator oligonucleotides may be usable by the template-independent DNA polymerase to generate the oligonucleotide or polynucleotide sequence(s).

In another aspect, the present disclosure provides devices for generating oligonucleotide or polynucleotide sequences. The devices may include a substrate, first electrode, second electrode, third electrode, a flow cell, or any combination thereof. The substrate may be configured to contact or may contact a solution comprising a template-independent DNA polymerase. The first electrode may be disposed on a surface of the substrate. The first electrode may be configured to immobilize or may immobilize initiator oligonucleotides usable by the template-independent DNA polymerase to generate the polynucleotide sequences. The first electrode may be configured to generate or may generate a local pH change to the solution disposed at a reaction site adjacent to the first electrode upon application of a first electrical potential to the first electrode. The second electrode may be disposed on the surface of the substrate adjacent to the first electrode. During use, the second electrode may be configured to confine or may confine the local pH change to the reaction site upon application of a second electrical potential to the second electrode. The third electrode may be disposed on the surface of the substrate adjacent to the first and second electrodes. During use the third electrode may be configured to permit or may permit measurement of current or voltage adjacent to the first electrode. The flow cell may be configured to deliver or may deliver the solution comprising the template-independent DNA polymerase to the substrate.

In another aspect, the present disclosure provides devices for large-scale synthesis of oligonucleotide or polynucleotide sequences for DNA data storage. The device may include a substrate, a plurality of initiator oligonucleotides, a flow cell, or any combination thereof. The substrate may comprise an array of reaction sites. A reaction site of the array of reaction sites may comprise a first electrode and a second electrode configured to generate a local pH change and confine the local pH change to the reaction site upon application of an electrical potential to the first electrode or the second electrode. The local pH change may be configured to activate a template-independent DNA polymerase. The reaction site may be individually addressable such that the template-independent DNA polymerase is usable to generate a plurality of unique polynucleotide sequences across the array of reaction sites. The plurality of initiator oligonucleotides may be immobilized to the array of reaction sites. The plurality of initiator oligonucleotides may be usable by the template-independent DNA polymerase to generate the plurality of unique polynucleotide sequences. The flow cell may be configured to provide or may provide the template-independent DNA polymerase to the substrate.

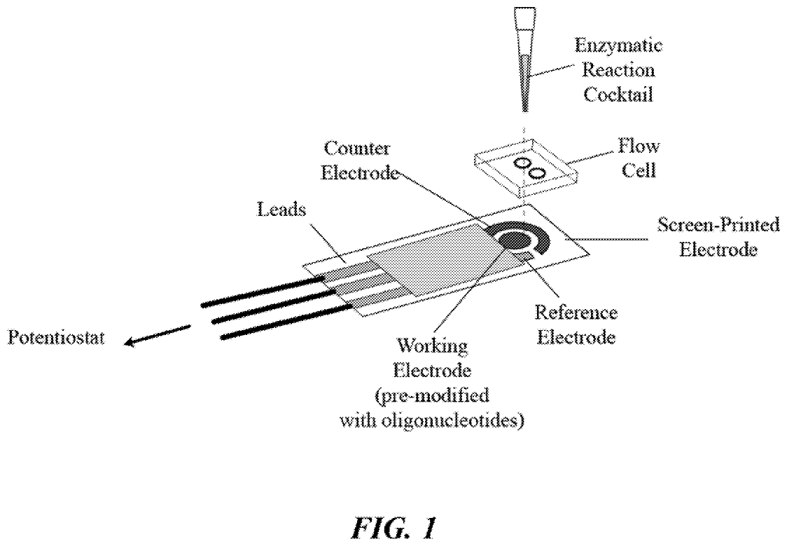

FIG. 1 shows an example device for electrochemical oligonucleotide or polynucleotide synthesis. The example device may include a reaction site that includes three electrodes. The electrodes may be screen-printed onto the substrate. The first electrode may be a circular planar electrode (e.g., working electrode) disposed in or near the center of the reaction site. The second electrode may partially circumscribe the first electrode. The second electrode (e.g., counter electrode) may be configured to electrically isolate the reaction site from another reaction site. The third electrode (e.g., reference electrode) may partially circumscribe the first electrode. The first electrode may be pre-modified with initiator oligonucleotides. The device may include circuitry configured to couple electroanalytical instrumentation (e.g., a potentiostat) to the device. The device may include three leads, one lead coupled to each of the electrodes. The device may include a flow cell configured to be in fluid communication with the reaction site. The flow cell may be configured to provide reaction mixture to the reaction site. As shown in FIG. 1, the example device may be coupled to a potentiostat. The potentiostat may provide a constant current to one or more of the electrodes. The current may stimulate a redox agent in the reaction mixture to generate protons and alter the pH of the reaction mixture within or near the reaction site.

The reaction site shown in FIG. 1 may be one of an array of reaction sites. A reaction site of an array of reaction sites may be configured to electrically isolate, physically isolate, or both a reaction site from another reaction site. The initiator oligonucleotides may be immobilized to the first electrode. A first electrode may be disposed on a substrate a distance from another first electrode, providing physical isolation of the reaction site. The second electrode at least partially circumscribing the first electrode may electrically isolate a reaction site from another reaction site. Physical and electrical confinement of the reaction sites may reduce or prevent cross-contamination between reaction sites. For example, physical confinement, electrical confinement, or both may prevent or reduce local pH changes in an individual reaction site from affecting the pH in another reaction site. Confinement of pH change to a reaction site may permit individual reaction sites to be individually addressable such that nucleotide incorporation to the initiator oligonucleotides at a reaction site may be controlled for the generation of target sequences.

Devices for oligonucleotide or polynucleotide synthesis may include a substrate. The substrate may comprise glass, metal, silicon, a polymer, or any combination thereof. A polymer substrate may include, but is not limited to, plastic, gel, resin, or any combination thereof. A substrate may have a surface area of greater than or equal to about 1 square millimeter (mm²), 2 mm², 4 mm², 6 mm², 8 mm², 10 mm², 15 mm², 20 mm², 25 mm², 30 mm², 40 mm², 50 mm², 60 mm², 70 mm², 80 mm², 90 mm², 100 mm², 125 mm², 150 mm², 175 mm², 200 mm², or more. A substrate may have a surface area of less than or equal to about 200 mm², 175 mm², 150 mm², 125 mm², 100 mm², 90 mm², 80 mm², 70 mm², 60 mm², 50 mm², 40 mm², 30 mm², 25 mm², 20 mm², 15 mm², 10 mm², 8 mm², 6 mm², 4 mm², 2 mm², 1 mm², or less. A substrate may have a surface area from about 1 mm² to 2 mm², 1 mm² to 4 mm², 1 mm² to 6 mm², 1 mm² to 8 mm², 1 mm² to 10 mm², 1 mm² to 15 mm², 1 mm² to 20 mm², 1 mm² to 25 mm², 1 mm² to 30 mm², 1 mm² to 40 mm², 1 mm² to 50 mm², 1 mm² to 60 mm², 1 mm² to 70 mm², 1 mm² to 80 mm², 1 mm² to 90 mm², 1 mm² to 100 mm², 1 mm² to 125 mm², 1 mm² to 150 mm², 1 mm² to 175 mm², 1 mm² to 200 mm², 2 mm² to 4 mm², 2 mm² to 6 mm², 2 mm² to 8 mm², 2 mm² to 10 mm², 2 mm² to 15 mm², 2 mm² to 20 mm², 2 mm² to 25 mm², 2 mm² to 30 mm², 2 mm² to 40 mm², 2 mm² to 50 mm², 2 mm² to 60 mm², 2 mm² to 70 mm², 2 mm² to 80 mm², 2 mm² to 90 mm², 2 mm² to 100 mm², 2 mm² to 125 mm², 2 mm² to 150 mm², 2 mm² to 175 mm², 2 mm² to 200 mm², 4 mm² to 6 mm², 4 mm² to 8 mm², 4 mm² to 10 mm², 4 mm² to 15 mm², 4 mm² to 20 mm², 4 mm² to 25 mm², 4 mm² to 30 mm², 4 mm² to 40 mm², 4 mm² to 50 mm², 4 mm² to 60 mm², 4 mm² to 70 mm², 4 mm² to 80 mm², 4 mm² to 90 mm², 4 mm² to 100 mm², 4 mm² to 125 mm², 4 mm² to 150 mm², 4 mm² to 175 mm², 4 mm² to 200 mm², 6 mm² to 8 mm², 6 mm² to 10 mm², 6 mm² to 15 mm², 6 mm² to 20 mm², 6 mm² to 25 mm², 6 mm² to 30 mm², 6 mm² to 40 mm², 6 mm² to 50 mm², 6 mm² to 60 mm², 6 mm² to 70 mm², 6 mm² to 80 mm², 6 mm² to 90 mm², 6 mm² to 100 mm², 6 mm² to 125 mm², 6 mm² to 150 mm², 6 mm² to 175 mm², 6 mm² to 200 mm², 8 mm² to 10 mm², 8 mm² to 15 mm², 8 mm² to 20 mm², 6 mm² to 25 mm², 8 mm² to 30 mm², 8 mm² to 40 mm², 8 mm² to 50 mm², 8 mm² to 60 mm², 8 mm² to 70 mm², 8 mm² to 80 mm², 8 mm² to 90 mm², 8 mm² to 100 mm², 8 mm² to 125 mm², 8 mm² to 150 mm², 8 mm² to 175 mm², 8 mm² to 200 mm², 10 mm² to 15 mm², 10 mm² to 20 mm², 10 mm² to 25 mm², 10 mm² to 30 mm², 10 mm² to 40 mm², 10 mm² to 50 mm², 10 mm² to 60 mm², 10 mm² to 70 mm², 10 mm² to 80 mm², 10 mm² to 90 mm², 10 mm² to 100 mm², 10 mm² to 125 mm², 10 mm² to 150 mm², 10 mm² to 175 mm², 10 mm² to 200 mm², 15 mm² to 20 mm², 15 mm² to 25 mm², 15 mm² to 30 mm², 15 mm² to 40 mm², 15 mm² to 50 mm², 15 mm² to 60 mm², 15 mm² to 70 mm², 15 mm² to 80 mm², 15 mm² to 90 mm², 15 mm² to 100 mm², 15 mm² to 125 mm², 15 mm² to 150 mm², 15 mm² to 175 mm², 15 mm² to 200 mm², 20 mm² to 25 mm², 20 mm² to 30 mm², 20 mm² to 40 mm², 20 mm² to 50 mm², 20 mm² to 60 mm², 20 mm² to 70 mm², 20 mm² to 80 mm², 20 mm² to 90 mm², 20 mm² to 100 mm², 20 mm² to 125 mm², 20 mm² to 150 mm², 20 mm² to 175 mm², 20 mm² to 200 mm², 25 mm² to 30 mm², 25 mm² to 40 mm², 25 mm² to 50 mm², 25 mm² to 60 mm², 25 mm² to 70 mm², 25 mm² to 80 mm², 25 mm² to 90 mm², 25 mm² to 100 mm², 25 mm² to 125 mm², 25 mm² to 150 mm², 25 mm² to 175 mm², 25 mm² to 200 mm², 30 mm² to 40 mm², 30 mm² to 50 mm², 30 mm² to 60 mm², 30 mm² to 70 mm², 30 mm² to 80 mm², 30 mm² to 90 mm², 30 mm² to 100 mm², 30 mm² to 125 mm², 30 mm² to 150 mm², 30 mm² to 175 mm², 30 mm² to 200 mm², 40 mm² to 50 mm², 40 mm² to 60 mm², 40 mm² to 70 mm², 40 mm² to 80 mm², 40 mm² to 90 mm², 40 mm² to 100 mm², 40 mm² to 125 mm², 40 mm² to 150 mm², 40 mm² to 175 mm², 40 mm² to 200 mm², 50 mm² to 60 mm², 50 mm² to 70 mm², 50 mm² to 80 mm², 50 mm² to 90 mm², 50 mm² to 100 mm², 50 mm² to 125 mm², 50 mm² to 150 mm², 50 mm² to 175 mm², 50 mm² to 200 mm², 60 mm² to 70 mm², 60 mm² to 80 mm², 60 mm² to 90 mm², 60 mm² to 100 mm², 60 mm² to 125 mm², 60 mm² to 150 mm², 60 mm² to 175 mm², 60 mm² to 200 mm², 70 mm² to 80 mm², 70 mm² to 90 mm², 70 mm² to 100 mm², 70 mm² to 125 mm², 70 mm² to 150 mm², 70 mm² to 175 mm², 70 mm² to 200 mm², 80 mm² to 90 mm², 80 mm² to 100 mm², 80 mm² to 125 mm², 80 mm² to 150 mm², 80 mm² to 175 mm², 80 mm² to 200 mm², 90 mm² to 100 mm², 90 mm² to 125 mm², 90 mm² to 150 mm², 90 mm² to 175 mm², 90 mm² to 200 mm², 100 mm² to 125 mm², 100 mm² to 150 mm², 100 mm² to 175 mm², 100 mm² to 200 mm², 125 mm² to 150 mm², 125 mm² to 175 mm², 125 mm² to 200 mm², 150 mm² to 175 mm², 150 mm² to 200 mm², or 175 mm² to 200 mm². In an example, the substrate has a surface area from about 1 mm² to 100 mm².

Devices for oligonucleotide or polynucleotide synthesis may include one or more electrodes. The electrodes may be flat, metallic electrodes. The electrodes may be coupled to an initiator oligonucleotide or a plurality of initiator oligonucleotides. The initiator oligonucleotide, or plurality of initiator oligonucleotides may be coupled to a surface of the electrode(s) by a covalent bond via thiol, amine, or other facile coupling chemistry. The initiator oligonucleotide, or plurality of initiator oligonucleotides, may be coupled to a surface of the electrode(s) via a linker molecule. The linker molecule may be configured to space the initiator oligonucleotide(s) further from the surface of the electrode(s) to improve enzyme accessibility and reaction efficiency.

The device may include a first electrode. The first electrode may be configured as or may be a working electrode. The first electrode may be configured as a cathode or an anode. In an example, the first electrode may be configured as a cathode. The first electrode may comprise a single material or may comprise multiple materials. The first electrode may be a flat, metallic electrode. In an example, the first electrode is a planar electrode. The first electrode may comprise platinum, copper, carbon, gold, aluminum, ruthenium, tungsten, silicon, silver, chromium, or any combination thereof. In an example, the first electrode comprises gold. In another example, the first electrode comprises gold and chromium. For example, gold electrodes may facilitate coupling of the initiator oligonucleotides to the electrodes via a thiol-gold bond.

The device may include a second electrode. The second electrode may be configured as or may be a counter electrode. The second electrode may be configured as a cathode or an anode. In an example, the second electrode may be configured as an anode. The second electrode may comprise a single material or may comprise multiple materials. The second electrode may be a flat, metallic electrode. In an example, the second electrode is a planar electrode. The first electrode and the second electrode may comprise the same materials. Alternatively, the first electrode and the second electrode may comprise different materials. The second electrode may comprise platinum, copper, carbon, gold, aluminum, ruthenium, tungsten, silicon, silver, chromium, or any combination thereof. In an example, the second electrode comprises gold. The second electrode may at least partially circumscribe the first electrode. The second electrode may be configured as an arc at least partially circumscribing the first electrode. The arc length of the second electrode may be at least about 25%, 30%, 40%, 50%, 60%, 70%, 80%, 90%, or more of a circle.

The device may include a third electrode. The third electrode may be configured as or may be a reference electrode. The third electrode may be configured to measure or may measure a response voltage or current of the solution in the reaction site. The third electrode may be a flat, metallic electrode. In an example, the third electrode is a planar electrode. The first electrode, second electrode, and third electrode may comprise the same material. Alternatively, the first electrode and the second electrode may comprise the same material and the third electrode may comprise a different material. The third electrode may comprise platinum, copper, carbon, gold, aluminum, ruthenium, tungsten, silver, chromium, or any combination thereof. In an example, the third electrode comprises silver. The third electrode may at least partially circumscribe the first electrode. The third electrode may be configured as an arc at least partially circumscribing the first electrode. The arc length of the third electrode may be less than about 50%, 40%, 30%, 25%, 20%, 15%, 10%, 5%, or less of a circle.

The electrode(s) (e.g., first, second, or third electrode) may be thin-film electrodes. The electrodes may be made of a single material or may comprise layers of different materials. The electrode(s) may be generated or otherwise manufactured by screen printing, electron-beam thin-film deposition, electrodeposition, thermal deposition, photolithography, inkjet printing, or any combination thereof. Any technique usable to generate thin films on a substrate may be used to generate an array of electrodes (e.g., reaction sites). The electrodes may have a thickness such that they protrude above a surface of the substrate. The electrodes (e.g., first, second, or third electrodes) may be the same thickness. Alternatively, the electrodes may have different thicknesses. For example, the first electrode may be thinner than the second electrode, the third electrode, or both. In another example, the first electrode may be thicker than the second electrode, the third electrode, or both. In another example, the electrodes have the same thickness. The thickness of the electrode(s) may be less than or equal to about 500 nanometers (nm), 400 nm, 300 nm, 250 nm, 200 nm, 150 nm, 100 nm, 75 nm, 50 nm, 40 nm, 30 nm, 25 nm, 20 nm, 15 nm, 10 nm, or less. The thickness of the electrode(s) may be from about 10 nm to 15 nm, 10 nm to 20 nm, 10 nm to 25 nm, 10 nm to 30 nm, 10 nm to 40 nm, 10 nm to 50 nm, 10 nm to 75 nm, 10 nm to 100 nm, 10 nm to 150 nm, 10 nm to 200 nm, 10 nm to 250 nm, 10 nm to 300 nm, 10 nm to 400 nm, 10 nm to 500 nm, 15 nm to 20 nm, 15 nm to 25 nm, 15 nm to 30 nm, 15 nm to 40 nm, 15 nm to 50 nm, 15 nm to 75 nm, 15 nm to 100 nm, 15 nm to 150 nm, 15 nm to 200 nm, 15 nm to 250 nm, 15 nm to 300 nm, 15 nm to 400 nm, 15 nm to 500 nm, 20 nm to 25 nm, 20 nm to 30 nm, 20 nm to 40 nm, 20 nm to 50 nm, 20 nm to 75 nm, 20 nm to 100 nm, 20 nm to 150 nm, 20 nm to 200 nm, 20 nm to 250 nm, 20 nm to 300 nm, 20 nm to 400 nm, 20 nm to 500 nm, 25 nm to 30 nm, 25 nm to 40 nm, 25 nm to 50 nm, 25 nm to 75 nm, 25 nm to 100 nm, 25 nm to 150 nm, 25 nm to 200 nm, 25 nm to 250 nm, 25 nm to 300 nm, 25 nm to 400 nm, 25 nm to 500 nm, 30 nm to 40 nm, 30 nm to 50 nm, 30 nm to 75 nm, 30 nm to 100 nm, 30 nm to 150 nm, 30 nm to 200 nm, 30 nm to 250 nm, 30 nm to 300 nm, 30 nm to 400 nm, 30 nm to 500 nm, 40 nm to 50 nm, 40 nm to 75 nm, 40 nm to 100 nm, 40 nm to 150 nm, 40 nm to 200 nm, 40 nm to 250 nm, 40 nm to 300 nm, 40 nm to 400 nm, 40 nm to 500 nm, 50 nm to 75 nm, 50 nm to 100 nm, 50 nm to 150 nm, 50 nm to 200 nm, 50 nm to 250 nm, 50 nm to 300 nm, 50 nm to 400 nm, 50 nm to 500 nm, 75 nm to 100 nm, 75 nm to 150 nm, 75 nm to 200 nm, 75 nm to 250 nm, 75 nm to 300 nm, 75 nm to 400 nm, 75 nm to 500 nm, 100 nm to 150 nm, 100 nm to 200 nm, 100 nm to 250 nm, 100 nm to 300 nm, 100 nm to 400 nm, 100 nm to 500 nm, 150 nm to 200 nm, 150 nm to 250 nm, 150 nm to 300 nm, 150 nm to 400 nm, 150 nm to 500 nm, 200 nm to 250 nm, 200 nm to 300 nm, 200 nm to 400 nm, 200 nm to 500 nm, 250 nm to 300 nm, 250 nm to 400 nm, 250 nm to 500 nm, 300 nm to 400 nm, 300 nm to 500 nm, or 400 nm to 500 nm.

The first electrode may be configured or otherwise shaped as a planar circle, ring, triangle, square, rectangle, or any other planar shape. In an example, the first electrode is configured or otherwise shaped as a planar circle. In another example, the first electrode is configured or otherwise shaped as a planar ring. The planer circle electrode may be disposed in or near the center of a reaction site. The second electrode may partially or completely circumscribe the first electrode. The second electrode may be configured to or may electrically isolate the reaction site from another reaction site. The initiator oligonucleotides may be immobilized on or adjacent to the first electrode. The initiator oligonucleotides may not be immobilized on or near the second electrode such that there is a physical separation (e.g., physical isolation) between initiator oligonucleotides immobilized on a first electrode and another first electrode. Physical and electrical isolation of the initiator oligonucleotides in the reaction site may prevent cross-contamination of reaction components between reaction sites.

The first electrode may be circumscribed, partially or fully, by the second electrode, third electrode, or combination thereof. The second electrode, third electrode, or both may be separated from the first electrode by a distance. The distance between the first and the second or third electrode may be at least about 10%, 12%, 30%, 40%, 50%, 60%, 80%, 100%, 120%, 140%, 160%, 200%, 250%, 300%, 400%, 500%, or more of a width of the first, second, or third electrode. For example, the second or third electrode may be a portion of a planar ring. The planar ring may have a width and may be positioned such that a spacing between the first electrode and the second or third electrode is from about 10% to 500% of a width of the planar ring of the second or third electrode.

The device may comprise an array of reaction sites. The array of reaction sites may include a plurality of first electrodes, a plurality of second electrodes, a plurality of third electrodes, or any combination thereof. A reaction site may include a first electrode and a second electrode. A reaction site may include a first electrode, a second electrode, and a third electrode. A first electrode may be shared between or associated with two or more reaction sites. Alternatively, or in addition to, a first electrode may not be shared between two or more reaction sites. A second electrode may be shared between or associated with two or more reaction sites. Alternatively, or in addition to, a second electrodes may not be shared between two or more reaction sites. A third electrode may be shared between or associated with two or more reaction sites. Alternatively, or in addition to, a third electrode may not be shared between two or more reaction sites. In an example, a reaction site includes a first electrode, a second electrode, and a third electrode none of which are shared with another reaction site. In another example, a reaction site includes a first electrode, a second electrode shared with another reaction site, and a third electrode.

An array of reaction sites may comprise a plurality of first electrodes and second electrodes. A ratio of first electrodes to second electrodes may be greater than or equal to about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or greater. A ratio of first electrodes to second electrodes may be less than or equal to about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, or less. In an example, a ratio of first electrodes to second electrodes is less than 1:1. In another example, a ratio or first electrodes to second electrodes is 1:1. In another example, a ratio of first electrodes to second electrodes is greater than 1:1. An array of reaction sites may comprise a plurality of separate second electrodes. Alternatively, or in addition to, an array of reaction sites may comprise one or more continuous second electrodes. For example, the second electrode may be configured as a grid of continuous electrodes.

A reaction site of the array of reaction sites may be individually addressable. For example, a first electrode of a reaction site may be individually addressable from another first electrode or another reaction site. A second electrode of a reaction site may be individually addressable from another second electrode of another reaction site. A third electrode of a reaction site may be individually addressable from another third electrode of another reaction site. In another example, a first and a second electrode of a reaction site may be individually addressable from another first electrode and another second electrode of another reaction site. In another example, a first, second, and third electrode of a reaction site may be individually addressable from another first, second, and third electrode of another reaction site. In an example, two or more reaction sites may share a second electrode and the first electrodes of the two or more reaction sites may be individually addressable.

The device may further include circuitry configured to couple to an electroanalytical device. Circuity may include one or more conductive pathways. In an example, each electrode may be coupled to an individual conductive pathway. Conductive pathways may be formed of the same materials as the electrode or different materials. The conductive pathways may comprise gold, silver, chromium, copper, aluminum, ruthenium, tungsten, or any combination thereof. In an example, the conductive pathways comprise silver. The electroanalytical device may include instrumentation usable for potentiometry, amperometry, conductometry, voltammetry, coulometry, or any combination thereof. The electroanalytical device may further supply power to the device. An electroanalytical device may be a potentiostat or galvanostat. The device may be configured for connection to an external electroanalytical device. Alternatively, or in additional to, the device may be configured to have or may have internal electroanalytical circuitry configured to measure electrical properties. In an example, the device may be coupled to a power supply and no measurement is performed.

The device may further include a flow cell configured to receive the solution and direct the solution to the substrate. The flow cell may include at least one inlet and at least one outlet. The inlet may be configured to receive or may receive a reaction mixture. The flow cell may be configured to provide the reaction mixture to the substrate, reaction site, or array or reaction sites. The outlet may be configured to remove the reaction mixture from the substrate, reaction site, or array of reaction sites. The flow cell may include valves, flow controller(s), sensors, or any combination thereof. The flow cell may be configured to minimize reagent volumes during synthesis operations to reduce overall synthesis costs. The flow cell may include a chamber that encompasses or otherwise encloses a portion of or all the surface area of an array of reaction sites. The flow cell may further include 1, 2, 3, 4, 5, or more channels configured to provide reagent(s) to the chamber, array of reaction sites, or both. In an example, the flow cell comprises a single channel configured to provide reagent(s) to the chamber, array of reaction sites, or both. The flow cell may be integrated with or coupled to one or more reservoirs. The one or more reservoirs may be configured to retain, or may retain, one or more solutions. The one or more solution may include reagent and reagent mixtures comprising nucleotide triphosphates, enzymes, buffers, additives, or any other reaction components described elsewhere herein. The one or more reservoirs may further include a wash solution configured to rinse the flow cell, array of reaction sites, or both.

The flow cell may be operatively coupled to a controller. The controller may be configured to direct or may direct the flow cell or reservoirs coupled thereto to provide one or more solutions to the chamber to contact the array of reaction sites. The controller may be configured to direct or may direct the flow cell to remove the one or more solutions from the chamber and array of reaction sites. The flow cell may be configured to re-use or otherwise recycle the solution(s) from a synthesis cycle in one or more subsequent synthesis cycles. The controller may be configured to direct or may direct a wash solution from a reservoir to the chamber and array of reaction sites to remove residual reaction products in preparation for subsequent nucleotide incorporation cycles.

The device may include an initiator oligonucleotide coupled to the substrate. In an example, the device may include a plurality of initiator oligonucleotides coupled to the substrate in or adjacent to the reaction site. Alternatively, or in addition to, the initiator oligonucleotides may be immobilized to the first electrode, the second electrode, the third electrode, or any combination thereof. In an example, the initiator oligonucleotides are immobilized to the first electrode and the second electrode. In another example, the initiator oligonucleotides are immobilized to the first electrode. An initiator oligonucleotide may comprise at least 1, 2, 3, 4, 5, 6, 8, 10, 12, 15, 20, or more nucleobases. The length of an initiator oligonucleotide may be longer than the minimum substrate length usable by the corresponding polymerase enzyme. In an example, an initiator oligonucleotide may comprise at least three nucleobases. In another example, the enzyme is a TdT polymerase, and the initiator oligonucleotide comprises at least three nucleobases. The plurality of initiator oligonucleotides may be the same across the array of reaction sites. Alternatively, or in addition to, different initiator oligonucleotides may be immobilized at different reaction sites. The initiator oligonucleotides may be immobilized via the 3′ end, 5′ end, or a combination thereof. In an example, the initiator oligonucleotides are immobilized via the 5′ end such that the 3′ end is enzymatically accessible.

The 5′ or 3′ end of the initiator oligonucleotides may comprise functionalization groups configured to couple the initiator oligonucleotides a surface of the reaction sites or to a linker molecule immobilized at the reaction sites. In an example, the 5′ end comprises a functionalization group on the 5′ end. Functionalization groups may include amines/acids for peptide coupling, alkynes/azides for copper “click” coupling, thiols/maleimides for thiol “click” coupling, biotin/streptavidin, or any combination thereof. The initiator oligonucleotides may be coupled directly to the surface of the substrate or the surface of an electrode. Alternatively, or in addition to, initiator oligonucleotides may be coupled to the substrate or electrode via linker molecules.

Initiator oligonucleotides or generated oligonucleotides or polynucleotides may comprise a primer sequence. The primer sequence may be a universal primer sequence or a targeted primer sequence. The primer sequence may be configured for downstream polymerase chain reaction (PCR) amplification, sequencing, or both.

Density of the initiator oligonucleotides may alter the activity of the template-independent DNA polymerase. For example, initiator molecules coupled to a reaction site at too high of a density may sterically hinder the template-independent DNA polymerase and reduce overall enzyme activity. Alternatively, initiator molecules coupled to a reaction site at too low of a density may diffusion limit the overall enzyme actively. The initiator oligonucleotides may be coupled to a reaction site at a density from about 10¹⁰ to 10¹¹ molecules per square centimeter (molecules/cm²), 10¹⁰ to 10¹² molecules/cm², 10¹⁰ to 10¹³ molecules/cm², 10¹⁰ to 10¹⁴ molecules/cm², 10¹⁰ to 10¹⁵ molecules/cm², 10¹¹ to 10¹² molecules/cm², 10¹¹ to 10¹³ molecules/cm², 10¹¹ to 10¹⁴ molecules/cm², 10¹¹ to 10¹⁵ molecules/cm², 10¹² to 10¹³ molecules/cm², 10¹² to 10¹⁴ molecules/cm², 10¹² to 10¹⁵ molecules/cm², 10¹³ to 10¹⁴ molecules/cm², 10¹³ to 10¹⁵ molecules/cm², or 10¹⁴ to 10¹⁵ molecules/cm². In an example, the initiator oligonucleotides may be coupled to a reaction site at a density from about 10¹⁰ to 10¹⁴ molecules/cm². A linker molecule may include polyethylene glycol (PEG) 200, PEG500, PEG1000, PEG2000, 11-mercaptoundecanoic acid (MUA), heterobifunctional linkers, polymeric linkers, or any combination thereof. In an example, the linker molecule is PEG1000. In another example, the linker molecule is MUA.

The linker molecules may be coupled to a surface of the reaction site via peptide coupling, alkyne-azide coupling, thiol-maleimide coupling, biotin-streptavidin coupling, silane-hydroxyl coupling, ester coupling, or any combination thereof. The linker molecules may comprise one or more functional groups configured to couple or that couple the linker molecules to a surface of the reaction site. In an example, the linker molecule includes a thiol-functionalization group. In another example, the linker molecules include aryldiazonium functionalization group.

The initiator oligonucleotides may be directly coupled to a surface of the substrate or an electrode (e.g., the first electrode or the first electrode and the second electrode). Alternatively, or in addition to, the initiator oligonucleotides may be coupled to a surface of the substrate or electrode via linker molecules. A linker molecule may be configured to control orientation or density of immobilized initiator oligonucleotides. A linker molecule may include a spacer. The spacer may be configured to space the initiator oligonucleotides away from a surface of the reaction site. Enzyme activity may be at least partially dependent upon spacer length. For example, directly coupling initiator oligonucleotides to the surface of an electrode (e.g., without a linker molecule and spacer) may reduce accessibility of the initiator oligonucleotides and reduce enzyme activity to well below solution phase enzyme activity. Alternatively, spacing the initiator oligonucleotides away from the surface of the substrate or electrode may permit enzyme activity to increase to solution phase enzyme activity or near to solution phase enzyme activity. Increasing spacer length may increase enzyme activity until solution phase enzyme activity is achieved. The length of the spacer may be at least about 0.2 nanometers (nm), 0.4 nm, 0.6 nm, 0.8 nm, 1 nm, 1.2 nm, 1.5 nm, 2 nm, 3 nm, 4 nm, 5 nm, 6 nm, 8 nm, 10 nm, or more. A length of the spacer may be less than or equal to about 10 nm, 8 nm, 6 nm, 5 nm, 4 nm, 3 nm, 2 nm, 1.5 nm, 1.2 nm, 1 nm, 0.8 nm, 0.6 nm, 0.4 nm, 0.2 nm, or less. The spacer may have a length of from about 0.2 nm to 0.4 nm, 0.2 nm to 0.6 nm, 0.2 nm to 0.8 nm, 0.2 nm to 1 nm, 0.2 nm to 1.2 nm, 0.2 nm to 1.5 nm, 0.2 nm to 2 nm, 0.2 nm to 3 nm, 0.2 nm to 4 nm, 0.2 nm to 5 nm, 0.2 nm to 6 nm, 0.2 nm to 8 nm, 0.2 nm to 10 nm, 0.4 nm to 0.6 nm, 0.4 nm to 0.8 nm, 0.4 nm to 1 nm, 0.4 nm to 1.2 nm, 0.4 nm to 1.5 nm, 0.4 nm to 2 nm, 0.4 nm to 3 nm, 0.4 nm to 4 nm, 0.4 nm to 5 nm, 0.4 nm to 6 nm, 0.4 nm to 8 nm, 0.4 nm to 10 nm, 0.6 nm to 0.8 nm, 0.6 nm to 1 nm, 0.6 nm to 1.2 nm, 0.6 nm to 1.5 nm, 0.6 nm to 2 nm, 0.6 nm to 3 nm, 0.6 nm to 4 nm, 0.6 nm to 5 nm, 0.6 nm to 6 nm, 0.6 nm to 8 nm, 0.6 nm to 10 nm, 0.8 nm to 1 nm, 0.8 nm to 1.2 nm, 0.8 nm to 1.5 nm, 0.8 nm to 2 nm, 0.8 nm to 3 nm, 0.8 nm to 4 nm, 0.8 nm to 5 nm, 0.8 nm to 6 nm, 0.8 nm to 8 nm, 0.8 nm to 10 nm, 1 nm to 1.2 nm, 1 nm to 1.5 nm, 1 nm to 2 nm, 1 nm to 3 nm, 1 nm to 4 nm, 1 nm to 5 nm, 1 nm to 6 nm, 1 nm to 8 nm, 1 nm to 10 nm, 1.2 nm to 1.5 nm, 1.2 nm to 2 nm, 1.2 nm to 3 nm, 1.2 nm to 4 nm, 1.2 nm to 5 nm, 1.2 nm to 6 nm, 1.2 nm to 8 nm, 1.2 nm to 10 nm, 1.5 nm to 2 nm, 1.5 nm to 3 nm, 1.5 nm to 4 nm, 1.5 nm to 5 nm, 1.5 nm to 6 nm, 1.5 nm to 8 nm, 1.5 nm to 10 nm, 2 nm to 3 nm, 2 nm to 4 nm, 2 nm to 5 nm, 2 nm to 6 nm, 2 nm to 8 nm, 2 nm to 10 nm, 3 nm to 4 nm, 3 nm to 5 nm, 3 nm to 6 nm, 3 nm to 8 nm, 3 nm to 10 nm, 4 nm to 5 nm, 4 nm to 6 nm, 4 nm to 8 nm, 4 nm to 10 nm, 5 nm to 6 nm, 5 nm to 8 nm, 5 nm to 10 nm, 6 nm to 8 nm, 6 nm to 10 nm, or 8 nm to 10 nm. In an example, the length of the spacer is greater than or equal to about 1.5 nm. In another example, the length of the spacer is less than or equal to about 8 nm.

Linker molecules or initiator oligonucleotides may include one or more cleavage sites configured to release the initiator oligonucleotide, oligonucleotide, or polynucleotide sequences from the reaction site. In an example, the linker molecules comprise one or more cleavage sites. In another example, the initiator oligonucleotides comprise one or more cleavage sites. The cleavage site may be configured to cleave or may cleave upon application an ultra-violet (UV) light, enzyme, electricity, heat, pH change, chemical reagents, or any combination thereof. In an example, the linker molecule may be cleaved by an enzyme. In another example, linker molecules or the initiator oligonucleotides comprise an uracil nucleobase cleavable by an Uracil-Specific excision reagent (USER). USER may include an Uracil DNA glycosylase and DNA glycosylase-lyase Endonuclease VIII. In another example, linker molecules of initiator oligonucleotides comprise photo-cleavable moieties cleavable by application of UV light.

The device may be usable for generating polynucleotide sequences for data storage. The device may be configured to store or otherwise encode information at a reaction site of the array of reaction sites. A reaction site may encode and store unique pieces of information. Alternatively, or in addition to, a reaction site may encode and store data that duplicates the data of another reaction site. The amount of information a device stores may be proportional to the number of unique polynucleotide sequences. A device may be configured to store or may store at least about 1000 bytes, 10,000 bytes, 100,000 bytes, 1 million bytes, 1.5 million bytes, 2 million bytes, 5 million bytes, 10 million bytes, 50 million bytes, 100 million bytes, 500 million bytes, 1 billion bytes, 2 billion bytes, 5 billion bytes, or more bytes of data. In an example, a device is configured to store or stores at least about 1 million bytes of data. In another example, a device is configured to store or stores at least about 1 billion bytes of data.

### Methods for Polynucleotide Synthesis

In an aspect, the present disclosure provides methods for generating oligonucleotide or polynucleotide sequences. The method may comprise providing a device comprising a substrate, a first electrode, a second electrode, a plurality of initiator oligonucleotides, or any combination thereof. The substrate may be in contact with a solution comprising a template-independent DNA polymerase. The first electrode may be disposed on a surface of the substrate and electrical communication with the solution. The second electrode may be disposed on the surface of the substrate and in electrical communication with the solution and the first electrode. The plurality of initiator oligonucleotides may be disposed in a reaction site adjacent to the first electrode. The method may further include applying an electrical potential to the first electrode or the second electrode to generate a local pH change to the solution in the reaction site. The local pH change may activate the template-independent DNA polymerase disposed in the reaction site. The method may further include using the plurality of initiator oligonucleotides and the template-independent DNA polymerase disposed in the reaction site to generate the polynucleotide sequences.

In another aspect, the present disclosure provides methods for generating oligonucleotide or polynucleotide sequences. The method may include providing a device. The device may include a substrate, a first electrode, a second electrode, a plurality of initiator oligonucleotides, or any combination thereof. The substrate may be in contact with a solution comprising a template-independent DNA polymerase. The first electrode may be disposed on a surface of the substate and in electrical communication with the solution. The second electrode may be disposed on the surface of the substrate and in electrical communication with the solution and the first electrode. The plurality of initiator oligonucleotides may be immobilized to a reaction site adjacent to the first electrode. The second electrode may at least partially circumscribe the first electrode. The method may further include applying a first electrical potential to the first electrode to generate a local pH change to the solution in the reaction site. The local pH change may activate the template-independent DNA polymerase disposed in the reaction site. The method may further include applying a second electrical potential to the second electrode to confine the local pH change to the reaction site. The method may further include using the plurality of initiator oligonucleotides and the template-independent DNA polymerase disposed in the reaction site to generate the polynucleotide sequences.

The methods described herein may be used with any of the devices or systems described elsewhere herein.

In an example, parallelized electrochemical oligonucleotide or polynucleotide synthesis may be performed by integrating an electrical source, electrodes modified with initiator oligonucleotides, a reaction mixture, or any combination thereof. The electrodes, as described elsewhere herein, may be disposed on a planer substrate. Reaction mixture may be added to coat or otherwise contact target areas. In the case of individual reaction sites, reaction mixture may be transferred directly to the reaction site. In the case of arrays of reaction sites, reaction mixture may be transferred to contact the entire array. Application of an electrical potential from an electrical source to the electrodes of a reaction site may result in oligonucleotide or polynucleotide synthesis. For arrays of reaction sites, reaction sites comprising in which electrical potential is applied may trigger synthesis whereas synthesis may not occur at reaction sites without electrical potential applied. Reaction sites without electrical potential applied may be inert toward the reaction mixture such that no nucleotide incorporation occurs. A potentiostat may be coupled to a configuration of three electrodes making up a reaction site.

Synthesis of oligonucleotide or polynucleotide sequences may include a series of cycles. A cycle may include incorporation of a unique nucleotide followed by a wash step. Incorporation may include a combination of incubating an array of reaction sites with a reaction mixture and application of an electrical potential to select reaction sites. A wash step may include an aqueous rinse of reaction sites to remove unincorporated nucleotides. For parallelized synthesis of multiple oligonucleotides or polynucleotides, logic may be incorporated to identify common bases between multiple sequences and performing simultaneous based addition across multiple sequences with a single synthesis cycle.

In an example, the polymerase may be a Mus musculus (M. musculus) TdT variant and the buffer may be a potassium phosphate buffer. The M. musculus TdT may switch from inactive or substantially inactive at a pH of 5.0 to active at a pH of 7. The pH change may be electrochemically initiated using a redox mediator (e.g., tetramethylquinone or tetramethylhydroquinone). The redox mediator may be reduced upon application of an electrical potential to the reaction site to release protons. In an example, release of protons into the reaction site may increase the buffer pH from approximately 5 to approximately 7. The electrodes may be configured such that the pH change is confined to the reaction site. For example, the relative position of the first and second electrodes may confine the pH change to the reaction site such that nucleotide incorporation may occur at a given reaction site and may not occur at another reaction site. This may permit selective initiation of nucleotide incorporation across an array of reaction sites via the selective application of electrical potential to a given reaction site.

Enzymatic polynucleotide synthesis methods described herein may use a template-independent polymerase, for example, terminal deoxynucleotidyl transferase (TdT) or other members of the X-family of DNA polymerases capable of template-independent activity, to catalyze the incorporation of nucleotide triphosphates (dNTP) onto single-stranded initiator oligonucleotides. Reaction components usable for incorporation of dNTPs into initiator oligonucleotides may include one or more of enzymes, oligonucleotide substrates, dNTPs, buffers, redox agents, salts, or any combination thereof. For surface- or chip-based synthesis initiator oligonucleotides may be immobilized, attached, or otherwise anchored on the surface of a substrate. A reaction mixture may be provided to the immobilized initiator oligonucleotides to permit incorporation of dNTPs and generation of the oligonucleotide or polynucleotide. Reaction efficiency of dNTP incorporation may be dependent upon initiator oligonucleotide orientation and immobilization density. Initiator oligonucleotide orientation and immobilization density may be tuned or otherwise modulated by surface chemistry, linker molecule length, and linker molecule type.

The method may use an electrode or a plurality of electrodes to modulate the activity of a polymerase enzyme. In an example, a reaction site may be a location on a substrate in which an extension reaction in is conducted. A reaction site may include a first electrode (e.g., working electrode), a second electrode (e.g., counter electrode), a third electrode (e.g., a reference electrode), or any combination thereof. The electrode or plurality of electrodes may be coupled to a potentiostat. The potentiostat may be configured to provide an electrical potential to one or more electrodes, monitor electrical environment of the reaction site, or both. An electrode (e.g., the first electrode) may be functionalized with a chemical linker (e.g., linker molecules) and an initiator oligonucleotide. Alternatively, or in addition to, the electrode (e.g., first electrode) may be functionalized with a plurality of chemical linkers (e.g., linker molecules) and a plurality of initiator oligonucleotides. A reaction mixture comprising one or more of a polymerase (e.g., TdT polymerase), buffer, a redox mediator or redox agent, or any combination thereof may be provided to the reaction site. The buffer and redox mediators may be selected such that the application of an electrical potential to the electrode(s) generates a change in local pH. For example, the application of an electrical potential to the reaction site may stimulate the redox mediator to release protons. The protons may generate a quantitative pH change in the reaction site, adjacent to the reaction site, or both. The change in local pH may be sufficient to activate or deactivate the polymerase. Activating or deactivating the polymerase may permit or prevent, respectively, nucleotide incorporation to the initiator oligonucleotide.

The method may further comprise generating or otherwise manufacturing an array of reaction sites. Generating the array of reaction sites may include screen printing, electron-beam thin-film deposition, electrodeposition, thermal deposition, photolithography, inkjet printing, or any combination thereof. In some examples, thin films may be generated by screen printing, electron-beam thin-film deposition, electrodeposition, or thermal deposition. The generated thin films may be patterned using photolithographic techniques. Any technique usable to generate thin films on a substrate may be used to generate an array of reaction sites. Each electrode may further comprise a conductive path (e.g., circuitry, conductive lead, etc.) configured to couple an electrode to electroanalytical instrumentation. The techniques described herein may be used to generate the conductive pathways.

The method may include immobilizing the plurality of initiator oligonucleotides to a surface of the reaction site or electrodes using various functionalization chemistries, such as thiol-gold functionalization. Alternatively, initiator oligonucleotides may be immobilized on a surface of a reaction site (e.g., electrode) via direct chemical functionalization. Initiator oligonucleotides may be immobilized onto a surface of the reaction site (e.g., electrode) via electrografting or other electrochemical functionalization techniques. Electrografting may include the use of any electrochemical reaction that permits organic (e.g., carbon containing) layers to be attached to solid conducting materials (e.g., electrodes). Electrografting may permit grafting of a chemical species to any conductive material. For example, an aryldiazonium group may be subjected to a reductive current/voltage to generate in situ a highly reactive aryl radical. The carbon radical may rapidly react and modify an adjacent two-dimension material (e.g., electrode surface). Aryldiazonium grafting may use a variety of radical-generating stimuli such as using materials with reducing properties, reducing reagents, high pH, photochemistry, or any combination thereof. Electrografting may provide an alternative to moiety specific surface functionalization techniques.

Alternatively, initiator oligonucleotides may be immobilized on the surface of the substrate or electrode(s) by moiety specific surface functionalization techniques. For example, the substrate may comprise gold and the functionalization reaction may include thiol-gold functionalization. Initiator oligonucleotides may be immobilized on the surface of the substrate or electrode using peptide coupling, alkyne-azide coupling, thiol-maleimide coupling, biotin-streptavidin coupling, or any combination thereof. In some examples, the aforementioned chemistries may be used to immobilize linker molecules on the surface. The immobilized linkers may be modified to include a free N-Hydroxysuccinimide (NHS), maleimide, azide, biotin, or another reactive moiety. The reactive moiety of the linker molecules may be reacted with a corresponding reactive moiety on the 5′ or 3′ end of the initiator oligonucleotides to immobilize the initiator oligonucleotides on the surface of the substrate or electrode. In an example, the plurality of initiator molecules is immobilized on the first electrode via spotting a solution comprising the linker molecules or the initiator oligonucleotides on the first electrode. Alternatively, the plurality of initiator molecules is immobilized on the first electrode via site specific chemistry (e.g., thiol-gold chemistry).

The method may include providing a plurality of nucleobases to the substrate. The nucleobases may be free nucleotides in their triphosphate form. The template-independent DNA polymerase may couple a nucleobase, or multiple nucleobases, of the plurality of nucleobases to initiator oligonucleotides disposed in the reaction site. The plurality of nucleobases may be of the same type, for example, the nucleobases may be cytosine, guanine, adenine, thymine, or uracil. The nucleobases may be provided to the substrate via a flow cell, electrical potential may be selectively applied to the reaction site(s) for a period of time, and, after the period of time, the electrical potential may be halted, the substrate may be washed to remove unincorporated nucleobases, and the next nucleobase may be provided to the substrate. The period of time may be greater than or equal to about 1 second, 30 seconds, 1 minute, 2 minutes, 3 minutes, 4 minutes, 5 minutes, 6 minutes, 8 minutes, 10 minutes or more. The period of time may be less than or equal to about 10 minutes, 8 minutes, 6 minutes, 5 minutes, 4 minutes, 3 minutes, 2 minutes, 1 minute, 30 seconds, 1 second, or less. The period of time may be from about 1 second to 30 seconds, 1 second to 1 minute, 1 seconds to 2 minutes, 1 seconds to 3 minutes, 1 seconds to 4 minutes, 1 seconds to 5 minutes, 1 seconds to 6 minutes, 1 seconds to 8 minutes, 1 seconds to 10 minutes, 30 second to 1 minute, 30 seconds to 2 minutes, 30 seconds to 3 minutes, 30 seconds to 4 minutes, 30 seconds to 5 minutes, 30 seconds to 6 minutes, 30 seconds to 8 minutes, 30 seconds to 10 minutes, 1 minute to 2 minutes, 1 minute to 3 minutes, 1 minutes to 4 minutes, 1 minute to 5 minutes, 1 minute to 6 minutes, 1 minute to 8 minutes, 1 minute to 10 minutes, 2 minutes to 3 minutes, 2 minutes to 4 minutes, 2 minutes to 5 minutes, 2 minutes to 6 minutes, 2 minutes to 8 minutes, 2 minutes to 10 minutes, 3 minutes to 4 minutes, 3 minutes to 5 minutes, 3 minutes to 6 minutes, 3 minutes to 8 minutes, 3 minutes to 10 minutes, 4 minutes to 5 minutes, 4 minutes to 6 minutes, 4 minutes to 8 minutes, 4 minutes to 10 minutes, 5 minutes to 6 minutes, 5 minutes to 8 minutes, 5 minutes to 10 minutes, 6 minutes to 8 minutes, 6 minutes to 10 minutes, or 8 minutes to 10 minutes. The time period may be the same for each nucleotide addition cycle or may vary for each nucleotide addition cycle. The nucleobases may be provided to the substrate sequentially until target sequences have been generated.

The nucleobases or nucleotides may be naturally occurring nucleotides, for example, deoxyadenosine triphosphate (dATP), deoxyguanosine triphosphate (dGTP), deoxycytosine (dCTP) triphosphate, deoxythymidine triphosphate (dTTP), or any combination thereof. The nucleotides may have modified or unmodified 5′ or 3′ hydroxyl groups. Nucleotides with unmodified 3′ hydroxyl groups may result in multiple additions of the same nucleotide (e.g., homopolymeric products). Oligonucleotides or polynucleotides with homopolymeric regions may be useful for DNA data storage applications where single base precision of synthesis may not be critical. In some cases, nucleotide with base modifications may be used to increase or decrease corresponding incorporation kinetics. Base modifications may include any modification, for example, 5′ dCTP modification, 5′ dTTP modifications, 3′ dGTP modifications, 3′ dATP modifications, or any combination thereof. Non-limiting examples of base modifications may include 5-hydroxymethyl, 5-propargylamino, 5-formyl, 5-carboxy, 5-hydroxy, 5-methyl, 5-iodo, 5-bromo, 3′-aminoxy, or any combination thereof. The use of modified bases may permit single nucleotides to be incorporated per incorporation cycle. Modified bases may include cleavable modifications such that the modification may be cleaved subsequent to incorporation of the modified base or subsequent to the incorporation of the next base in the oligonucleotide or polynucleotide sequence. Cleavable base modifications may include 3′-aminooxy, 3′-O-azidomethyl, 3′-O-allyl, or 3′-O-nitrobenzyl dNTPs. In some examples, the modification is a blocking moiety that prevents the incorporation of subsequent nucleotides. The blocking moiety may be removed to generate a free 3′ hydroxyl and permit subsequent nucleotide incorporation. The use of blocking moieties and other moieties to control or otherwise modulate nucleotide incorporation may permit controlled nucleotide incorporation. Precise control over nucleotide incorporation may provide for generation of targeted oligonucleotide or polynucleotide sequences that may be useful in a variety of applications, including life science applications.

The reaction mixture use may be such that the template-independent DNA polymerase is inactive in the bulk solution. The pH of the reaction mixture may be less than or equal to about 11, 10, 9, 8, 7, 6, 5, 4, or less. In an example, the pH of the reaction mixture is less than or equal to about 6. In another example, the pH of the reaction mixture is less than or equal to about 5.

The local pH of the reaction mixture disposed in a reaction site may increase upon application of an electrical potential to the electrodes (e.g., the first electrode, second electrode, or both). The template-independent DNA polymerase may be activated by the increase in pH in the reaction site. Alternatively, the local pH of the reaction mixture disposed in the reaction site may decrease upon application of an electrical potential to the electrodes (e.g., the first electrode, second electrode, or both). The template-independent DNA polymerase may be activated by the decrease in pH in the reaction site. The template-independent DNA polymerase may be active when disposed adjacent to a reaction site in which an electrical potential is being applied to the electrodes of the reaction site. The local pH of the reaction mixture may be greater than or equal to about 4, 5, 6, 7, 8, 9, or more upon application of an electrical potential to the electrodes of a reaction site. In an example, the local pH of the reaction mixture in the reaction site is greater than or equal to 6 upon application of an electrical potential to the electrodes of a reaction site. In another example, the local pH of the reaction mixture in the reaction site is greater than or equal to 7 upon application of an electrical potential to the electrodes of a reaction site.

Halting or otherwise ceasing application of the electrical potential to the electrode(s) of a reaction site may return the local pH back to the bulk pH. Returning the local pH in or adjacent to the reaction site to the bulk pH may inactivate the template-independent DNA polymerase.

The reaction mixture may include a redox agent or mediator. The redox agent or mediator may transduce electrical inputs into a corresponding pH change. The redox agent or mediator may act as an electrochemically-driven pH switch to toggle enzyme activity between active and inactive. The redox agent or mediator may permit or provide for the local pH change of the solution in the reaction site during application of an electrical potential (e.g., application of a current or voltage to one or more electrodes). Application of an electrical potential to the electrode(s) of the reaction site may generate an oxidation reaction that produces protons to decrease the local pH in the reaction site. Redox agents or mediators may undergo oxidation to liberate protons and lower the pH or reduction to consume protons and increase the pH. In some examples, reduction and oxidation events may free or consume hydroxide ions, also changing the pH. The redox agent or mediator may be orthogonal in biochemistry in that it does not otherwise interfere with other biochemical reaction components. The redox agent or mediator may be water soluble or suitably soluble to dissolve via a co-solvent. Redox agents or mediators that are non-polar may first be dissolved in DMSO or similar solvents prior to adding to an aqueous reaction mixture. The redox agent or mediator may include tetramethylquinone, tetramethylhydroquinone, substituted quinones or hydroquinones, water, or any combination thereof. In an example, the redox agent may be tetramethylquinone. Upon application of an electrical potential, tetramethylquinone may undergo reduction to consume protons and yield tetramethylhydroquinone, as shown in Equation 1.

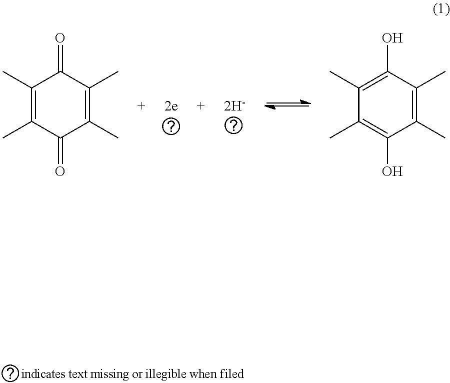

The reaction mixture may include a buffer with an equivalence point at or near the pH dependency point of the template-independent DNA polymerase. At the equivalence point, a change in proton concentration may permit a large change in pH, for example, on the order of 0.5, 1, 2, 3, or more. Application of an electric current to the reaction mixture comprising a redox agent or mediation may cause the pH of the solution (e.g., local solution) to shift by at least 0.5, 1, 2, 3, or more change in pH. The buffer may have an equivalence point at a pH of about 4, 5, 6, 7, or 8. The buffer may include potassium phosphate, sodium carbonate, physiological buffers, or any combination thereof. Physiological buffers may include bicarbonate buffers, hemoglobin buffers, phosphate buffers, protein buffers, or ammonium buffers. Buffers may include sodium, potassium and lithium cations. Buffers may further comprise divalent cations (e.g., magnesium) that may compete with divalent ions added to the reaction mixture. The buffer may be a strong electrolyte to support electrochemistry. Buffers comprising zwitterionic buffers (e.g., MES, HEPES, etc.) may not be as effective as buffers that easily dissociate at supporting electrochemistry.

The method may comprise providing a template-independent DNA polymerase with the reaction mixture to a reaction site or array of reaction sites. The enzyme for enzymatic DNA synthesis may be capable of facilitating de novo formation of oligonucleotides or polynucleotides. The template-independent DNA polymerase may include terminal deoxynucleotidyl transferase (TdT), which is a DNA polymerase that is able to extend single-stranded oligonucleotides at the 5′ end. The polymerase (e.g., TdT polymerase) may be a naturally occurring or an engineered polymerase. In an example, the TdT is from Mus musculus. The enzyme activity of the template-independent DNA polymerase may sharply vary as a function of pH. For example, the enzyme activity may change from inactive to active, or active to inactive, over a pH range of less than or equal to about 0.5, 1, 2, 3, or 4. At pH values of less than or equal to about 8, 7, 6, 5, 4, or less the enzyme activity may be negligible or inactive. At pH values of greater than or equal toa bout 4, 5, 6, 7, 8, or more the enzyme activity may be high. Alternatively, enzyme activity may be negligible or inactive at a higher pH (e.g., pH 7 or greater) and active at a lower pH (e.g., pH of 7 or lower). In an example, the enzyme may undergo a transition in activity from a pH of about 4 to 5, 4 to 6, 4 to 7, 4 to 8, 5 to 6, 5 to 7, 5 to 8, 6 to 7, 6 to 8, or 7 to 8. In an example, the enzyme may undergo a transition in activity from a pH of about 5 to 7. The enzyme formulation (e.g., following protein expression and purification) may not include species that may interfere with surface-based synthesis, for example, thiols or beta-mercaptoethanol which may disrupt thiol-functionalized gold surfaces. Recombinant enzyme (e.g., TdT) may be purified in a combination of Tris buffer, sodium chloride, and glycerol.

The reaction mixture may include cofactors, such as divalent ions. Divalent ions may be used by the template-independent DNA polymerase to for incorporation of nucleotides into oligonucleotide or polynucleotide sequences. The reaction mixture may include magnesium, manganese, cobalt, zinc, or any combination thereof. In an example, the reaction mixture includes magnesium. In another example, the reaction mixture includes magnesium and manganese. The same cofactors may be added to each reaction cycle (e.g., each time a type of nucleotide is added). Alternatively, the cofactors may be tuned based on the nucleotide to be incorporated. For example, manganese may be a cofactor usable for dCTP or dCTP analog incorporation.

The method may further comprise using a third electrode to measure a response voltage or current of the solution in the reaction site. Measuring the response voltage or current may permit the applied electrical potential to be modulated in real time to maintain local pH change.

The method may further comprise cleaving the oligonucleotide or polynucleotide sequences from the array of reaction sites. Cleavage may be targeted to oligonucleotides or polynucleotides at select reaction sites. Alternatively, oligonucleotides or polynucleotides may be non-specifically cleaved across the array of reaction sites. The cleaved oligonucleotides or polynucleotides may include a primer sequence. The method may further include amplifying the oligonucleotide or polynucleotide sequences via the primer sequence. The primer sequence may be the same for oligonucleotides or polynucleotides at the different reaction sites. Alternatively, the primer sequence of the oligonucleotides or polynucleotides may be unique to a reaction site such that amplification of sequences from a given reaction site may be targeted. In an example, amplification may be surface based amplification. In another example, the sequences may be cleaved, and amplification may be solution phase amplification. Amplification may include polymerase chain reaction, isothermal amplification, ligation-mediated amplification, or any combination thereof.

The method may further comprise sequencing the oligonucleotides or polynucleotides using the primer sequences. Initiator oligonucleotides or generated oligonucleotides or polynucleotides may comprise a primer sequence. The primer sequence may be a universal primer sequence or a targeted primer sequence. The primer sequence may be configured for downstream polymerase chain reaction (PCR) amplification, sequencing, or both. Sequencing the oligonucleotides or polynucleotides may comprise sequencing by synthesis, sequencing by hybridization, or any combination thereof.

The method may further comprise generating oligonucleotide or polynucleotide sequences for encoding data. Encoded information may be represented by a plurality of bit sequences. Encoding data may include assigning a bit value (e.g., zero or one) to a nucleobase or a sequence of nucleobases. A bit sequence may be encoded by generating a plurality of polynucleotide sequences at a reaction site. A polynucleotide sequence may encode information such that a reaction site encodes a unique piece of information. Alternatively, multiple reaction sites may encode the same piece of information. The method may further include decoding encoded information. The information may be decoded by sequencing or otherwise detecting the sequence of the polynucleotide sequences.

### Computer Systems

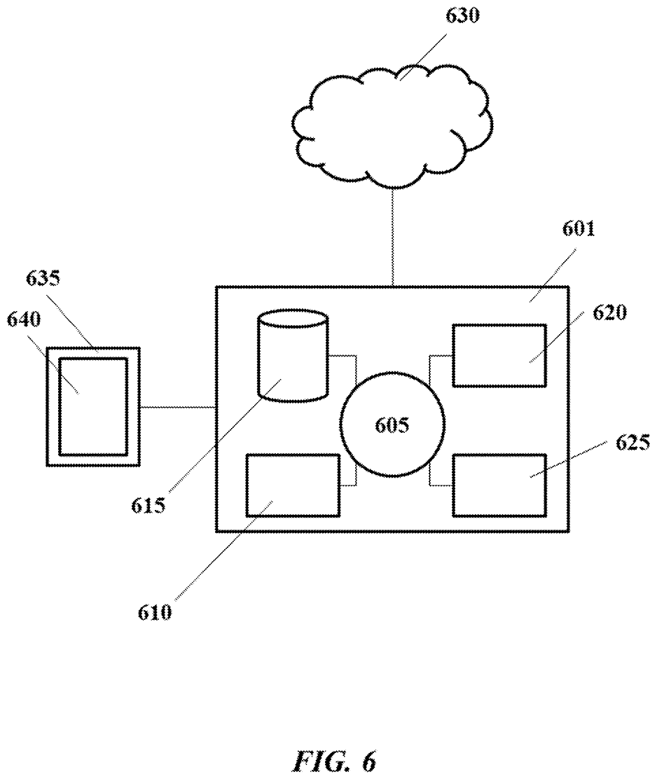

The present disclosure provides computer systems that are programmed to implement methods of the disclosure. FIG. 6 shows a computer system 601 that is programmed or otherwise configured to synthesize nucleic acid molecules. The computer system 601 can regulate various aspects of the methods, systems, and devices of the present disclosure, such as, for example, regulating current delivered to the electrodes to control nucleic acid synthesis. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., technician, scientist, or another user). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, status of nucleic acid synthesis, flow rates, current or voltage at or around the electrodes, or any combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, control processes described elsewhere herein, including flow rate of fluids provided to an electrode array, current or voltage provided to the electrodes, or any combination thereof.

### EXAMPLES

### Example 1: Preparing Oligonucleotide Functionalized Gold-Films

Application of electricity to the electrodes (e.g., first and second electrode or working and counter electrode) may increase or otherwise turn on terminal deoxynucleotidyl transferase (TdT) activity via changes in local pH. Increasing TdT activity may permit enzymatic polynucleotide synthesis. In this example, the relationship between pH and enzymatic activity is determined.

Gold-coated microscope slides having 500 angstroms (Å) of gold and 50 A of chromium are purchased from Angstrom Engineering. The gold-coated slides are cut into small pieces with a scribe and soaked in an ethanolic solution in the presence of a thiol-functionalized polyethylene glycol (PEG) linker (HS-PEG-COOH, molecular weight (MW) 1000 g/mol, purchased from Creative PEGWorks) for 1 hour to couple the PEG linker to the gold coating and provide a free carboxyl group. The concentration of the PEG linker in the ethanol solution is 100 millimolar (mM). The microscope pieces are rinsed with ethanol and dried. The dried pieces are covered by a solution of 200 mM 1-ethyl-3-[3-dimethylaminopropyl]carbodiimide (EDC) and 100 mM N-hydroxysuccinimide (NHS) in 50 mM pH 6 2-[N-morpholino] ethanesulfonic acid (MES) buffer and incubated for 1 hour in a humidified chamber to generate a succinimide-functionalized surface for coupling with amine-containing molecules. After incubation, the pieces are rinsed with ultrapure water, dried, and placed in a desiccant container. In the desiccant container, the pieces are spotted via pipette with 1-2 microliters (μL) of a solution of 10 μM amino-terminated oligonucleotide (purchased from IDT Technologies at a concentration of 100-500 μM in ultrapure water) in 0.1 molar (M) pH 9 cesium carbonate buffer for 1 hour. After immobilization of the oligonucleotide via “drop drying” the pieces are rinsed with water, Triton-X (0.1% v/v aqueous solution), and water again and dried. The pieces are incubated in a secondary wash buffer of 100 mM lithium chloride (LiCl), 0.1 mM ethylenediaminetetraacetic acid (EDTA), 0.1% Triton-X, and 50% glycerol for 10 minutes to remove free (e.g., un-immobilized) oligonucleotide molecules. Microscope pieces are rinsed with water and dried.

### Example 2: pH Tuning of Surface-Based Terminal Deoxynucleotidyl Transferase Activity

To perform on-chip enzymatic synthesis, a biochemical reaction mixture comprising 5.1 μL of ultrapure water, 1 μL potassium phosphate buffer, 1.3 μL of 5 mM divalent salt (e.g., magnesium acetate, manganese chloride), 1.3 μL of 1 mM free deoxynucleotide triphosphate (dNTP) solution, 1.3 μL of a 4 milligrams per milliliter (mg/mL) TdT polymerase solution is pipetted on the surface of the microscope pieces and incubated for six minutes. After incubation, the surface is rinsed with water, Triton-X (0.1% v/v aqueous solution), and water again, then dried.

The deoxynucleotide triphosphate may be dATP, dCTP, dTTP, dGTP, or modified deoxynucleotide triphosphates. The TdT may be the M. musculus variant of TdT polymerase. The enzyme is recombinantly expressed in Escherichia coli with a polyhistidine tag and purified via affinity chromatography. The final storage buffer for the enzyme is 20 mM tris hydrochloride, 100 mM LiCl, and 50% glycerol at pH 7.3.

The M. musculus variant of TdT polymerase may have high activity in potassium phosphate buffer at pH 7. The combination of potassium phosphate buffer at pH 7, divalent salt (e.g., manganese²⁺) and dNTP (e.g., dCTP) may yield long homopolymeric polynucleotide products.

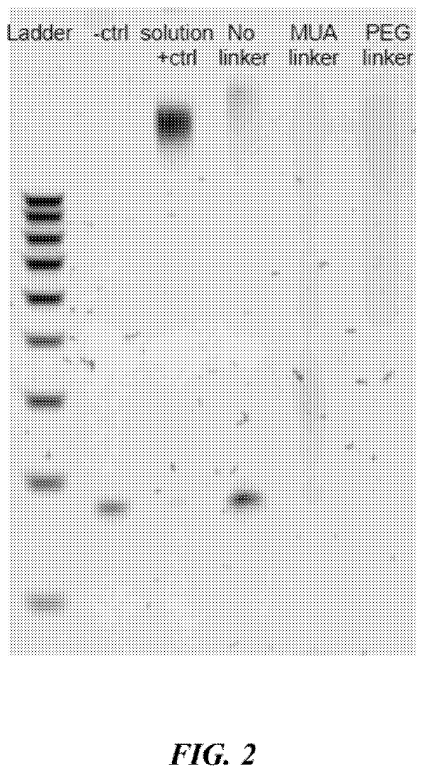

On surface activity of TdT may be less than solution phase activity of TdT. As shown in FIG. 2, linker length may alter the surface-based TdT activity. Short linkers, such as 11-mercaptoundecanoic acid linker, may reduce TdT activity below that of solution phase activity. Longer linkers, such at 1000 MW PEG, main increase the on surface TdT activity to close to that of solution phase TdT activity.

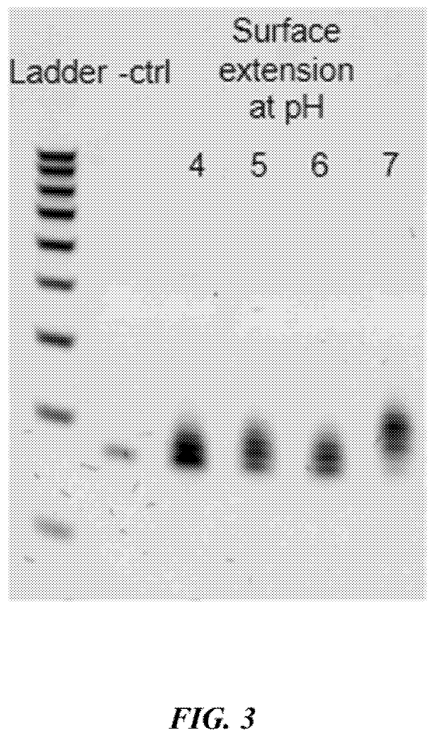

To determine the effect of pH on surface-based TdT activity, potassium phosphate buffer at various acid pH's (e.g., 4, 5, 6, and 7) were used with the above described biochemical reaction mixture. FIG. 3 shows enzyme activity, as measured by the length of TdT extension products on a TBE-Urea gel. As shown in FIG. 3, addition of thymine bases via 5-hydroxymethyl dUTP and magnesium acetate, which is a combination that yields short homopolymeric polynucleotide products, does not occur at pH 4 and 5, some extension occurs at pH 6, and complete extension occurs at pH 7.

### Example 3: Electrochemical Nucleic Acid Synthesis

Electrochemical nucleic acid synthesis is demonstrated using 220BT gold screen-printed electrodes (SPEs) from Metrohm DropSens as an example electrode system. The example electrodes use a gold first electrode (e.g., working electrode) that is 4 millimeters (mm) in diameter surrounded by a gold second electrode (e.g., counter electrode) and a silver third electrode (e.g., reference electrode. The SPEs include silver leads for coupling to a potentiostat. Oligonucleotides are immobilized on the gold electrodes using the process described in Example 1. Briefly, a thiol PEG linker is immobilized on the gold electrodes. The thiol PEG linker is subjected to an acid-to-succinimide conversion followed by immobilization of an amine-terminated oligonucleotide.

Two SPEs are affixed in a square acrylic mold via double-sided tape to generate a flow cell. The flow cell encircles the entire three-electrode region and includes an inlet and outlet hole to permit pipette addition of fluid to the flow cell. The silver leads of one of the two SPEs are connected to the corresponding wires of a handheld potentiostat (PalmSens4, from PalmSens).

A reaction mixture is pipetted into the flow cell for both SPEs. The reaction mixture includes tetramethylquinone (TMQ, purchased from Sigma-Aldrich) and tetramethylhydroquinone (TMHQ, purchased from TCI America) dissolved in DMSO as a redox mediator. The reaction mixture is generated by combining 8.2 μL ultrapure water, 2 μL potassium phosphate buffer at pH 5, 2.6 μL of a 5 mM divalent salt (e.g., magnesium acetate), 2.6 μL 1 mM dNTP, 2.6 μL of a 4 mg/ml TdT polymerase, and 2 μL of a 30 mM one-to-one TMQ-to-TMHQ in DMSO for a total volume of 20 μL.

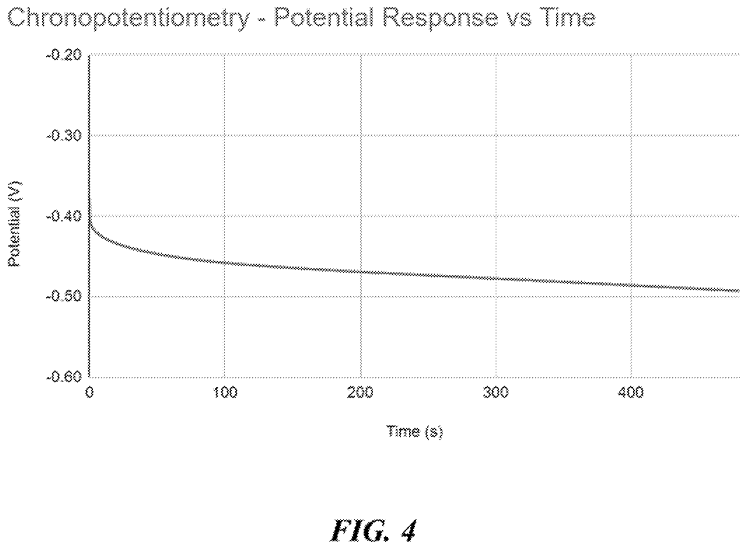

A reductive current of −5 microamperes (μA) is applied to the SPE via the potentiostat using a “chronopotentiometry” method as the electrode region is incubated with the reaction mixture. After application of the current, the flow cell is rinsed with water, Triton-X (0.1 v/v % aqueous solution, water again, and dried. The flow cell may be disassembled for rinsing. Electrochemical response of the SPE may be confirmed using the potentiostat's potential versus time graph, as shown in FIG. 4. Over the course of electrochemical synthesis, the potential is maintained between −0.4 and −0.5 volts (V). Electrochemical synthesis may be confirmed through surface oligonucleotide cleavage and gel analysis.

### Example 4: Analysis of Polynucleotide Synthesis Products

Products of the surface-based polynucleotide synthesis are cleaved off of the surface of the SPEs using enzymatic treatment with uracil DNA glycosylase (USER) enzyme (New England Biosciences). The immobilized single stranded DNA (ssDNA) includes an uracil cleavage site that is a target for the USER enzyme. Cleavage of the ssDNA at the cleavage site releases the 3′-portion of the oligonucleotide into solution. The USER mixture is prepared by combining 1 μL of 1× CutSmart buffer, 1 μL of 1U/μL USER enzyme, and 8 μL of ultrapure water for a total volume of 10 μL. The USER mixture is pipetted onto the gold-coated pieces or SPEs that have undergone polynucleotide synthesis. The gold-coated pieces or SPEs are incubated at 37 °C for 30 to 45 minutes. Water is added to the chip and the resultant fluid is collected via pipette to recover the synthesized oligonucleotides. An aliquot of recovered oligonucleotide fluid is analyzed via gel electrophoresis on a 15% TBE-Urea gel (Novex/ThermoScientific).

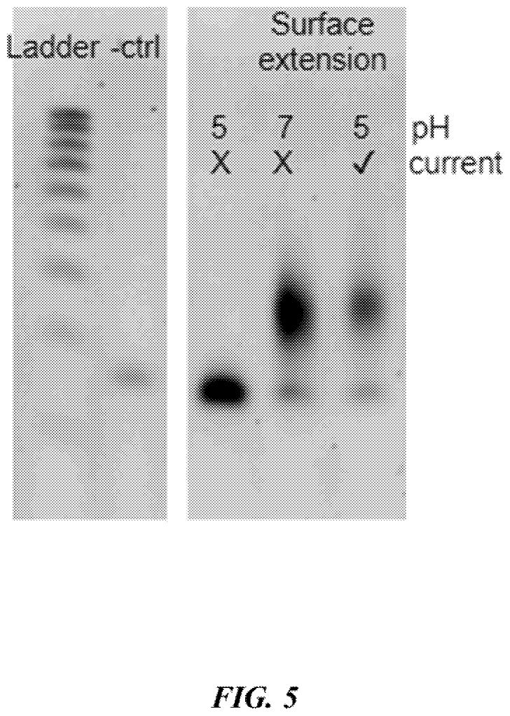

As shown in FIG. 5, electrochemical synthesis on the SPE results in a higher molecular weight band similar to that of a control (e.g., non-electrochemical) synthesis on a separate SPE. The SPE that does not have a voltage applied results in no nucleotide incorporation (e.g., no band shift), showing that electrodes in which voltage is applied selectively undergo polynucleotide synthesis while other electrodes remain unchanged. By iterating this electrochemical synthesis process with different deoxynucleotide triphosphates, multiple unique polynucleotide sequences may be generated.

### Example 5: Polynucleotide Data Storage

In an example, the devices and methods described herein are usable for encoding and storing data. Encoded information is represented by a plurality of bit sequences. Encoding may include assigning a bit value (e.g., zero or one) to a nucleobase. A bit sequence may be encoded by generating a plurality of polynucleotide sequences at a reaction site. The plurality of polynucleotide sequences may encode the information in the bit sequence such that each reaction site encodes a unique piece of information. The information may be decoded by sequencing or otherwise detecting the sequence of the generated polynucleotide sequences.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A device for generating polynucleotide sequences, comprising:
a substrate configured to contact a solution comprising a template-independent deoxyribonucleic acid (DNA) polymerase;
a first electrode disposed on a surface of the substrate, wherein the first electrode is configured to be in electrical communication with the solution;
a second electrode disposed on the surface of the substrate, wherein the second electrode is configured to be in electrical communication with the solution, wherein the first electrode and the second electrode are configured to generate a local pH change to the solution in a reaction site adjacent to the first electrode upon application of an electrical potential to the first electrode or the second electrode, and wherein, during use, the local pH change activates the template-independent DNA polymerase disposed in the reaction site; and
a plurality of initiator oligonucleotides immobilized on the first electrode by a plurality of linker molecules such that, during use, the plurality of initiator oligonucleotides is disposed in the reaction site, and wherein the plurality of initiator oligonucleotides is usable by the template-independent DNA polymerase to generate the polynucleotide sequences.

2. The device of claim 1, wherein a linker molecule of the plurality of linker molecules comprises a spacer.

3. The device of claim 2, wherein a length of the spacer is greater than or equal to about 1.5 nanometers (nm).

4. The device of claim 3, wherein the length of the spacer is less than or equal to about 8 nm.

5. The device of claim 1, wherein a linker molecule of the plurality of linker molecules comprises a cleavage site.

6. The device of claim 5, wherein the cleavage site comprises an uracil nucleobase.

7. The device of claim 5, wherein the cleavage site is configured to be cleaved upon application of an ultra-violet (UV) light, enzyme, electricity, heat, pH, or any combination thereof.

8. The device of claim 1, wherein the plurality of linker molecules comprise one or more members selected from the group consisting of polyethylene glycol (PEG) 200, PEG500, PEG1000, PEG2000, 11-mercaptoundecanoic acid, heterobifunctional PEG linkers, and any combination thereof.

9. The device of claim 1, wherein the first electrode comprises one or more members selected from the group consisting of gold, aluminum, ruthenium, tungsten, silicon, and any combination thereof.

10. The device of claim 9, wherein the plurality of linker molecules comprises thiol-functionalization groups.

11. The device of claim 1, wherein the plurality of linker molecules comprises aryldiazonium functionalization group.

12. The device of claim 1, wherein the plurality of linker molecules is coupled to the first electrode via peptide coupling, alkyne-azide coupling, thiol-maleimide coupling, biotin-streptavidin coupling, or any combination thereof.

13. The device of claim 1, further comprising a third electrode configured to measure a response voltage or current of the solution in the reaction site.

14. The device of claim 1, wherein the device comprises a plurality of first electrodes comprising the first electrode and a plurality of second electrodes comprising the second electrode.

15. The device of claim 14, wherein the first electrode and second electrode of the reaction site are individually addressable from another first electrode and another second electrode of another reaction site.

16. The device of claim 1, further comprising circuitry configured to couple to an electroanalytical device.

17. The device of claim 1, further comprising a flow cell configured to receive the solution and direct the solution to the substrate.

18. The device of claim 1, wherein an initiator oligonucleotide of the plurality of initiator oligonucleotides comprises at least three nucleobases.

19. The device of claim 1, wherein an initiator oligonucleotide of the plurality of initiator oligonucleotides is coupled to the first electrode via a 5′ end.

20. The device of claim 1, wherein the first electrode and the second electrodes are planar electrodes.

21-64. (canceled)
